(19) United States
(12) Patent Application Publication
(10) Pub. No.: US 2017/0239428 A1
(43) Pub. Date: Aug. 24, 2017

(54) VAPOR MEDICINE DISPENSING AND NANO-MIST LUNG CLEANSER SYSTEM WITH NASAL ADAPTER

(71) Applicant: Nano Mist, LLC, Los Angeles, CA (US)

(72) Inventor: Ike Banoun, Los Angeles, CA (US)

(21) Appl. No.: 15/422,872

(22) Filed: Feb. 2, 2017

Related U.S. Application Data
(60) Provisional application No. 62/289,992, filed on Feb. 2, 2016.

(51) Int. Cl. A61M 11/00; A61M 16/10; A61M 39/24; A61K 31/045; A61K 31/4045; A61K 9/00; A61K 36/35; A61K 31/122; A61K 31/192; A61K 31/519; A61K 36/53; A61M 16/06; A61K 31/135

(52) U.S. Cl. CPC A61M 11/00; A61M 16/06; A61M 16/109; A61M 39/24; A61K 31/045; A61K 31/4045; A61K 31/135; A61K 36/35; A61K 31/122; A61K 31/192; A61K 31/519; A61K 36/53; A61K 9/0078; A61M 2205/8206; A61M 2210/1039; A61M 2205/75

(57) ABSTRACT

A system, accompanying apparatuses and methods relating to an oral, nasal or propellant enhanced mist delivery, drug delivery and lung cleansing apparatuses and having for an object to provide a safe and harmless means for and method of delivering medication with heated, moist, flavored air; or by inhaling warm medication like methanol into the lungs in case of a respiratory ailment under direction of a physician. Additionally, all state of the art medicines are envisioned for delivery with the system.

### CROSS REFERENCE TO RELATED APPLICATION

This application takes priority from and claims the benefit of U.S. Provisional Application Ser. No. 62/289,992, the contents of which are herein incorporated by reference.

### FIELD OF THE INVENTION

The present system relates to medical systems for delivery of drugs, medications, cleansers, heated, moist, or flavored air.

### BACKGROUND OF THE INVENTION

Drug delivery refers to approaches, formulations, technologies, and systems for transporting a pharmaceutical compound in the body as needed to safely achieve its desired therapeutic effect. It may involve scientific site-targeting within the body, or it might involve facilitating systemic pharmacokinetics; in any case, it is typically concerned with both quantity and duration of drug presence. Drug delivery is often approached via a drug's chemical formulation, but it may also involve medical devices or drug-device combination products. Drug delivery is a concept heavily integrated with dosage form and route of administration, the latter sometimes even being considered part of the definition. Concurrent drug delivery technologies modify drug release profile, absorption, distribution and elimination for the benefit of improving product efficacy and safety, as well as patient convenience and compliance. Drug release is from: diffusion, degradation, swelling, and affinity-based mechanisms. Most common routes of administration include the preferred non-invasive perioral (through the mouth), topical (skin), trans mucosal (nasal, buccal/sublingual, vaginal, ocular and rectal) and inhalation routes.

Many medications such as peptide and protein, antibody, vaccine and gene based drugs, in general may not be delivered using these routes because they might be susceptible to enzymatic degradation or cannot be absorbed into the systemic circulation efficiently due to molecular size and charge issues to be therapeutically effective. For this reason many protein and peptide drugs have to be delivered by injection or a nano-needle array.

For example, many immunizations are based on the delivery of protein drugs and are often done by injection. Current efforts in the area of drug delivery include the development of targeted delivery in which the drug is only active in the target area of the body (for example, in cancerous tissues) and sustained release formulations in which the drug is released over a period of time in a controlled manner from a formulation. In order to achieve efficient targeted delivery, the designed system must avoid the host's defense mechanisms and circulate to its intended site of action. Types of sustained release formulations include liposomes, drug loaded biodegradable microspheres, and drug polymer conjugates.

### SUMMARY OF THE INVENTION

The instant apparatus, system and accompanying methods, as illustrated herein, is clearly not anticipated, rendered obvious, or even present in any of the prior art mechanisms, either alone or in any combination thereof. Therefore, it is an object of the instant invention to provide various systems and accompanying apparatuses for oral, nasal or propellant mist enhanced delivery and lung cleansing system and associated apparatuses.

The present system, accompanying apparatuses and methods relate to an oral, nasal or propellant enhanced mist delivery and lung cleansing apparatuses and has for an object to provide a safe and harmless means for and method of delivering medication with heated, moist, flavored air; or by inhaling warm medication like methanol into the lungs in case of a respiratory ailment under direction of a physician.

It is also an objective to introduce a system including a primary use of life extension and pain management. Another object of the system is to provide an article of manufacture wherein air may be drawn through a porous substance or filter mechanism of a cartridge which has been moistened with a chemically harmless flavoring preparation.

An additional objective of the instant system is to combine moisture and taste wherein the moist and flavored air passes through a section of the device heated by a suitable heating element in order that warm, moist and flavored air is drawn into the mouth of the user, and if desired, into the lungs of the user.

A further object of the instant system is to provide a nano-vaporization system smokeless for circulating the fluid around the heating element in a turbulent manner to suitably raise the temperature of the inhalant mixture, with the purpose that the temperature of the flavored air.

A further object is to insulate the heat source as the mechanism may be too hot for the user to hold as well as to introduce a nasal adapter device to be utilized with instant vapor medicine delivery system, which can be adapted for both human and animal applications. An additional object is to introduce a nano-misting or nano-mist system, which can be adapted for both human and animal applications.

A further object of the instant system is to introduce an apparatus with the capability of delivering water, menthol, eucalyptus or glycerol beads of a micro size when compared to current vaporizers by Vicks® or other such systems which deliver large water drops that don't go deep in the lungs. Moreover, concurrent inhalers and nebulizers do not deliver droplets measured in nanometers.

An added and prominent objective of the instant device is to introduce a face mask to prevent transmission of disease and a filtration system to protect against potential contamination. And, an unexpected result of experimentation with drug delivery processes focuses on the positive utilization of the by-product vapor alone to clean, clear and soothe the lungs and enhance lung functions with the clean steam with small particle size.

An unexpected result of nicotine vaporizers is that the vapor can carry beneficial drugs that actually treat diseases as well as administer treatments and therapies. Unlike today's existing technology which is used as a cigarette cessation or replacement product containing the carcinogen nicotine and in no way is it used or meant to be for the benefits, promotion of health in fact curing or pain managements here cited. You are in effect steam cleaning your lungs and thus entails a whole new entry point for these drugs into the body. Also introduced, a plain water vapor, with nothing added, from these devices, was observed to open the lungs and allow the user to breathe better.

The varied the size of vapor droplets to suit the molecule size of drugs using the existing technology heat control can lead to immediate migraine treatment with such drugs Sumitriptan®. It was not expected that existing devices can work for headaches and other pain management, such as NSAIDs for arthritis.

In additional embodiments, the system may be design to utilize pharmaceutical prescribed drugs (other not nicotine) and caffeine may be help reduce the boiling point or volatilization of another drug. It can also be used for energy vapor markets to replace the energy drink space. Unexpected was that these devices deposit drugs under tongue and in all mouth receptors so delivery is not only through lungs. Different than current use which is intended for lung delivery.

Further addressing the nasal vaporizing as another point of entry totally different than concurrent systems, this would be beneficial to treat many things nasally as well as another entry point to the lungs that is not done using current tech. Also, replaces nasal spray.

Intranasal is a good area for low dose potent drugs, since surface area is normally too small. An additional route for vaccines since you need very little to obtain an antigenic immune response. Thus the instant system allows the user to administer gene therapy as well as viruses and vaccines through the lungs.

Additionally, the system may be utilized for diagnostics and may deliver small molecule drugs as deep as this into the lungs. The depth and reach of drug deposition is unique as the stomach is bypassed stomach for efficacy, purity and speed and thus the drugs or medicines are inherently purer. Thus, mixtures or cocktails can be blended. For example, adding caffeine for energy and to lower the vaporizing temp for other drugs that will not survive the heat.

Aromatherapy usage can clearly provide more focused attention and can be part of our nutritional or neutraceuticals. Wine and alcohol mixed with flavors and glycol or other carriers. Any and all compounds and mixtures may be utilized to cleanse lungs, including common menthol. Sublingual, and mouth and lung combination absorption is possible, including B12 absorption. The vaped B12 will be absorbed thru the mouth and the lungs providing extra energy to the body.

Further, nacetyl lcysteine may be vaped into the lungs and combining this with a nonionic surfactant like polysorbate 80 (TWEEN 80) to create the best available compound which will also be mucolytic. Additives for infections may also be utilized. Thus, all nutraceuticals may be employed, again because the process is in liquid form. And a further combination of mucolytic drugs, cysteine and a non-ionic surfactant (TWEEN 80, poysorbate or other non-ionic surfactants can also be used. The system can therefore replace nasal spray because it delivers a micro-aerosolized dose of therapeutic medication.

Thus, the instant system to deliver drugs and other actives via various routes where other delivery systems have failed or require injections. The delivery system allows drugs to be vaporized (vaped) and efficiently delivered to the lungs, through the nasal route and mouth, including sublingual and across other mucosal membranes. The target market includes delivering efficiently nutraceuticals, herbals, prescription and non-prescription drugs including vitamins, vaccines, anti-pyretics, pain (narcotic and non-narcotic including migraine headaches), diabetes, cystic fibrosis, drugs for obesity and anti-ageing, in a vaporized state. Several patents have been filed to include over-the-counter (OTC) drugs, nutraceuticals, prescription drugs such as nitroglycerin, sildenafil (Viagra, and other similar drugs), active ingredients of Vicks VapoRub and recreational and lifestyle style improving drugs. All drugs in a solvent system that can be vaporized and are heat stable under the E-cig temperatures are included. Vaccines can be delivered in developing countries in a cost-effective manner without cross contamination by changing the disposable tip attached to the device.

Additionally, in one embodiment, the instant device will be unique for medicine as it will comprise a one-way fill valve and thus operate as a one way inhaler to prevent spread of disease if device is being shared, and HEPA® and other filters or better. The fill valve will be spring-loaded may be torsion or other such mechanism as well and which allows the cartridge to be filled with a “needle-point” bottle. The cartridge may be a disposable tip with a one way valve.

For the instant system, this will allow for the cartridge to be filled with medication, while preventing the “patient” from extracting the solution from the cartridge. A specific voltage may to be preset on the battery device specific for each medication, along with an automatic shut-off feature preventing the solution from reaching a defined maximum temperature.

In additional embodiments, steroids, testosterone alcohol, cortisone, prednisone may be administered. Further and even blowing it out in a fine mist to cure large areas of the outside of the body. Can administer burn and many therapies or actual drugs like Botox. Furthermore, a vaporizing vaccine is introduced and also the clearance of lungs due to Propylene Glycol, using Camphor in small particle emission and even vaping of antibiotics to treat pneumonia rather quickly, and a process for treating lung disease with herbs.

The instant system can thus transport antitussive herbs to reduce respiratory spasms; expectorant herbs loosen mucus; demulcent herbs sooth irritated tissue; and antimicrobial herbs resolve infections. For transportation of drugs, in the instant system they may not have to be frozen as they are submerged in the VG/PG or other carrier, thus buried and protected. Also process patents for the drugs that have never been processed in this manner. The drugs are processed through the lungs, not IV, Pills, Powder inhalers, or nebulizers. The system also incorporates sublingual administration as the vapor effects all receptors of the mouth.

Moreover, many processes for the treating of disease and introducing drugs are herein illustrated, for example the system may be utilized to assist breathing for asthma patients. Further, in any of the mixture, the addition of a small quantity of water in the mix to, can prevent dehydration a recurring occurrence with VG/propylene glycol non-aqueous systems. The quantity of the water will be adjusted so that we do not get precipitation of the active. If required, additional ingredients will be added such that the system delivers with a clear mixture or a colored mixture as required for medical or diagnosis purposes. Thus, the instant system represents discovered a new way to improve the current VG/PG mix and transport of all known drugs using an enveloping solution like PG/VG.

Further, Lavender oil, which possesses a scent with a calming effect which may aid in relaxation and the reduction of anxiety and stress may be employed. Lasea capsules containing Lavender oil with a high amount of linalool and linayl acetate termed as Silexan by the manufacturer are approved as an anxiolytic in Germany. The approval is based on the finding that the capsules are comparable in effect to low-dose lorazepam. Additionally, all of the systems herein are adaptable to veterinary use.

Moreover, the instant system may cover all parenteral and non-parenteral routes for administering drugs. The parenteral routes include intravenous, intramuscular, subcutaneous and intradermal. The non-parenteral routes include oral, sublingual, buccal, nasal and vaginal. Drugs such as Bayer®, baby aspirin, and other drugs for migraine and headache, as well as, Pfizer® Viagra® when delivered sublingually map provide biological stability and rapid absorption of the drug.

Yet another object of the present system is to provide a lung cleansing treatment that cleans and clears lungs due to its depth of reach into the smallest airways. Lung cleansing treatment works because the size of the droplet created by the current system is measured in nanometers, and droplets at this size can go to the smallest of airways in the lungs, where menthol and other ingredients inside these particles can be delivered. The droplets are able to attach themselves to the cold receptors in the lining of the airways, which creates shrinkage. The skin retracts as the menthol opens the airways, creating greater breathing capacity. The smallest size of the droplets is key for reaching areas in hard to reach places within the lungs. Most inhaled corticoid drug particles, due to their large size, only make it to half of the lungs, and most of the powder stays in the throat. The nano-particles created by the current system are thus so small in stature as to penetrate the alveoli, and thus proceed where powdered drugs are unable.

The current system generates a mist that as stated is composed of tiny droplets in the low nanometer range (menthol in a gaseous state). The menthol is soothing and cooling in a gaseous state that on inhalation reaches the deep innermost portions of the lungs.

There has thus been outlined, rather broadly, the more important features of the versatile systems, apparatuses and accompany methods in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carries out in various ways. Also, it is to be understood that the phraseology and terminology employed herein are for the purpose of description and should not be regarded as limiting.

These, together with other objects of the invention, along with the various features of novelty, which characterize the invention, are pointed out with particularity in the claims annexed to and forming a part of this disclosure. For a better understanding of the interactive systems, apparatuses and accompany methods, the operating advantages and the specific objects attained by usage, reference should be made to the accompanying drawings and descriptive matter in which there are illustrated preferred embodiments of the invention.

### BRIEF DESCRIPTION OF THE DRAWINGS

The invention may be more completely understood in consideration of the following detailed description of various embodiments of the invention in connection with the accompanying drawings, in which:

FIG. 1 illustrates a front view of one embodiment of the nasal adapter utilizing a face mask mechanism;

FIG. 2 illustrates a side view of one embodiment of the nasal adapter utilizing a face mask mechanism;

FIG. 3 illustrate an exploded view of one embodiment of the nasal adapter and face mask mechanism;

FIG. 4 illustrates a side view of one embodiment of the nasal adapter utilizing a face mask mechanism;

FIG. 5 illustrate a top view of one embodiment of the nasal adapter utilizing a face mask mechanism;

FIG. 6 illustrates an exploded view of a face mask mechanism in use;

FIG. 7 illustrates a side view of one embodiment of the nasal adapter with the face mask mechanism in use; and

FIG. 8 illustrates a side view of one embodiment of the nasal adapter utilizing the face mask mechanism in use.

### DETAILED DESCRIPTION OF THE INVENTION

The detailed description set forth below is intended as a description of presently preferred embodiments of the apparatus and does not represent the only forms in which the present apparatus may be construed and/or utilized. The description sets forth the functions and the sequence of the steps for producing the system and accompanying apparatus. However, it is to be understood that the same or equivalent functions and sequences may be accomplished by different embodiments also intended to be encompassed within the scope of the invention.

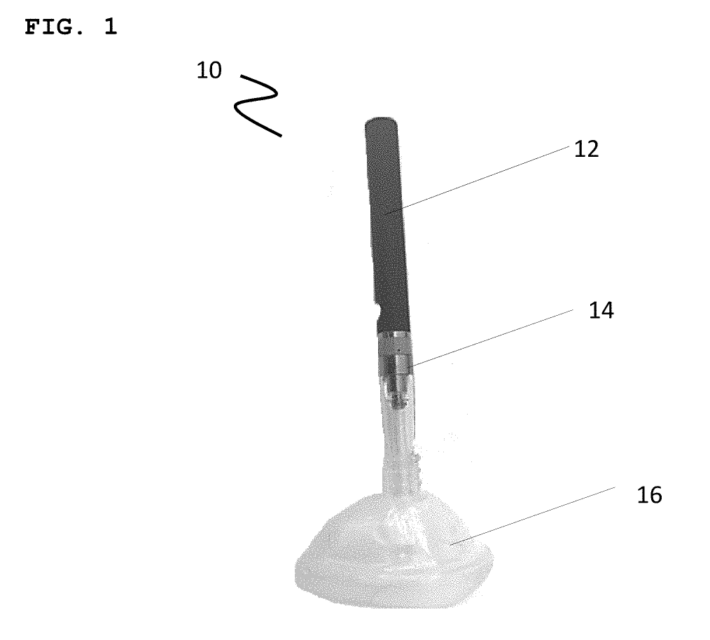

Therefore, turning to the drawings, FIG. 1 illustrates a front view of one embodiment of a nasal adapter utilizing a face mask mechanism. The nasal adapter 10 may comprise a rechargeable battery device 12, an atomizer 14, and a face mask mechanism 16. In a preferred embodiment, the rechargeable battery 12 provides a power source wherein when activated the power source heats a liquid within the atomizer 14 and vapor is generated when the heated liquid makes contact with the atomizer 14. In a preferred embodiment, the atomizer utilizes the vaporized lung cleansing system from pending U.S. Pat. No. 1,4704,506

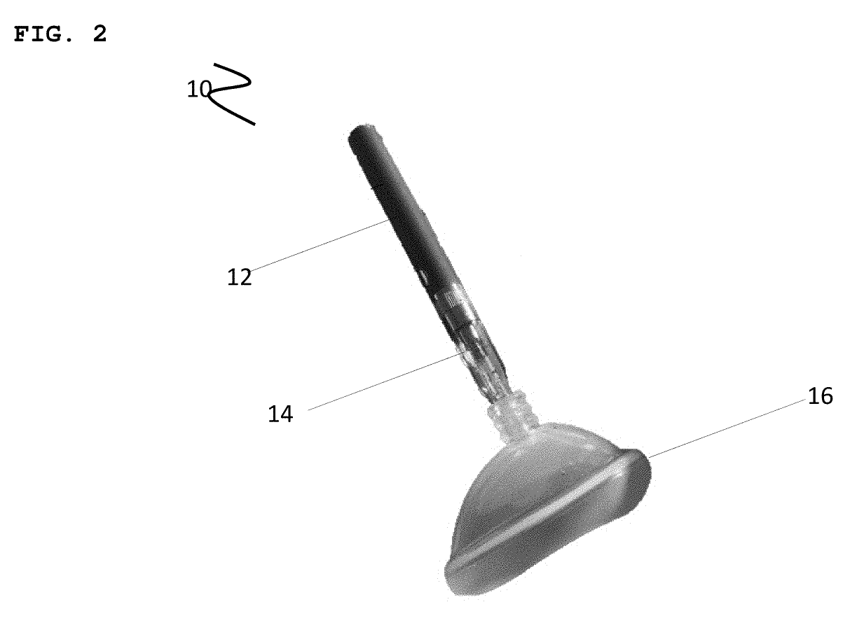

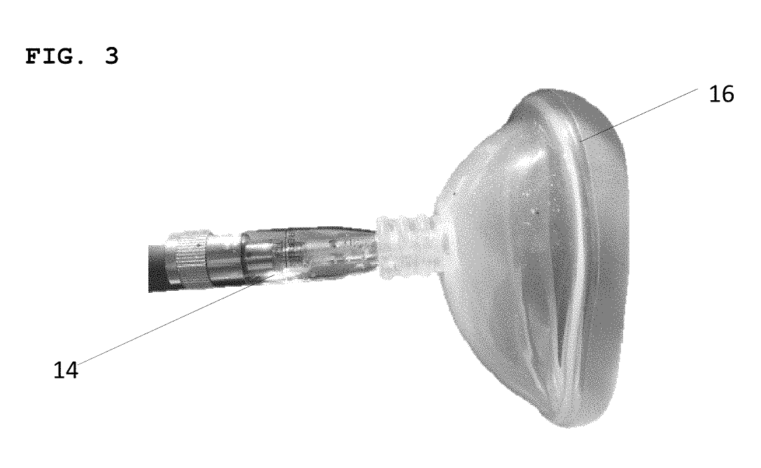

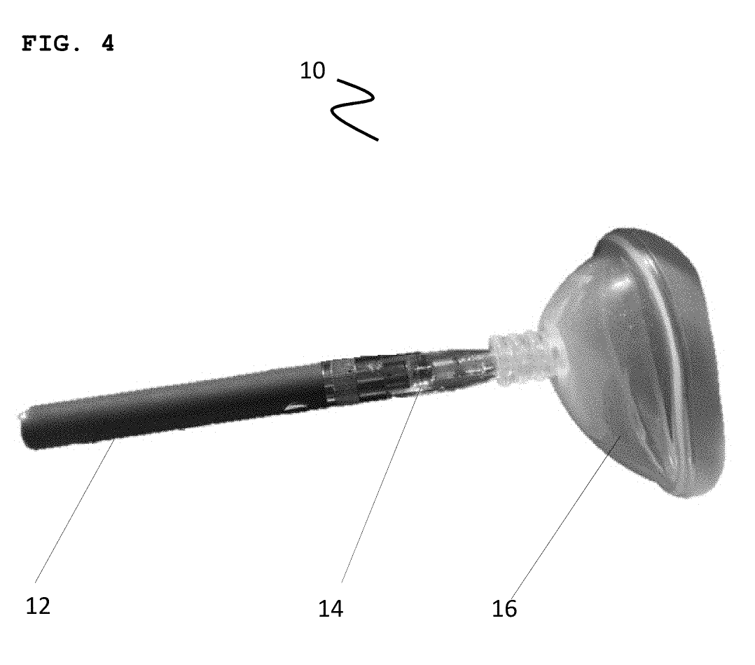

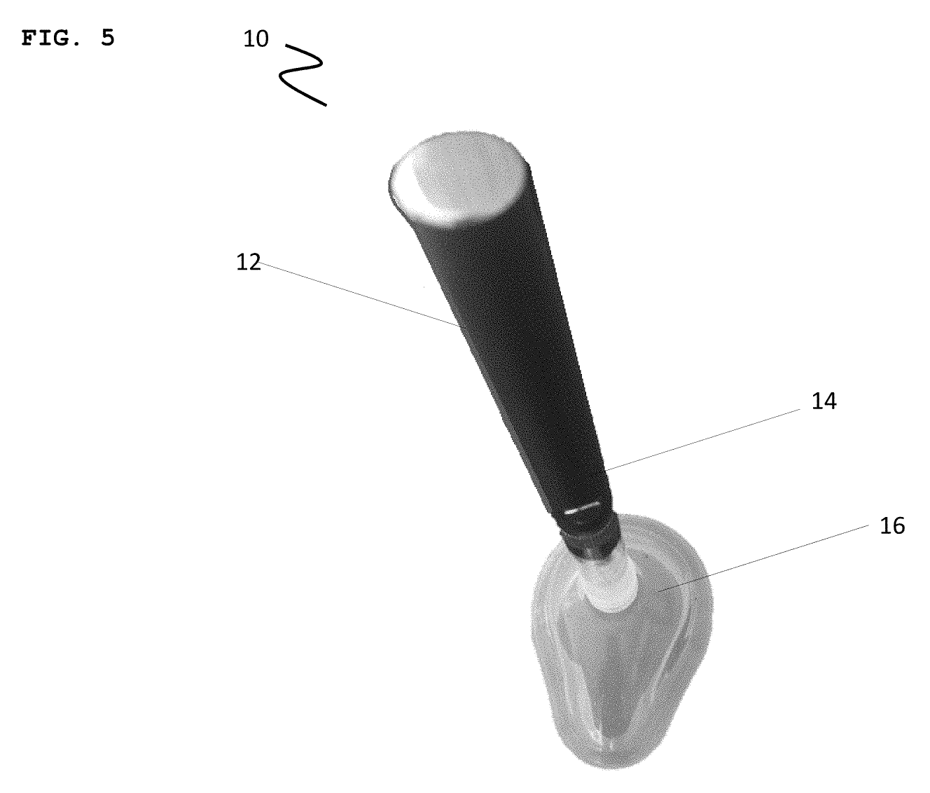

FIG. 2 illustrates a side view of the nasal adapter 10 utilizing a face mask mechanism 16. FIG. 3 illustrates an exploded view of the face mask mechanism 16 and an exploded view of the atomizer 14. FIG. 4 is another side view of the nasal adapter 10 and accompanying face mask 16. FIG. 5 illustrates a top view of the nasal adapter 10.

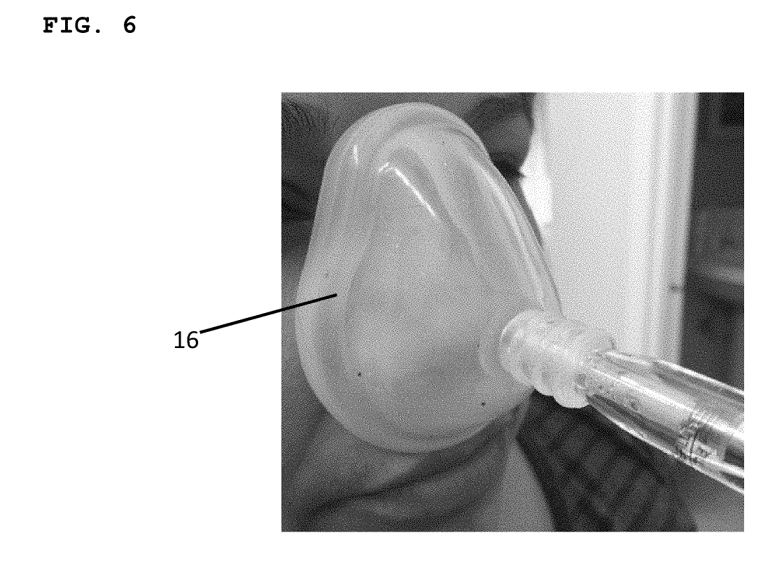

FIG. 6 illustrates an exploded view of the face mask mechanism 16 in use.

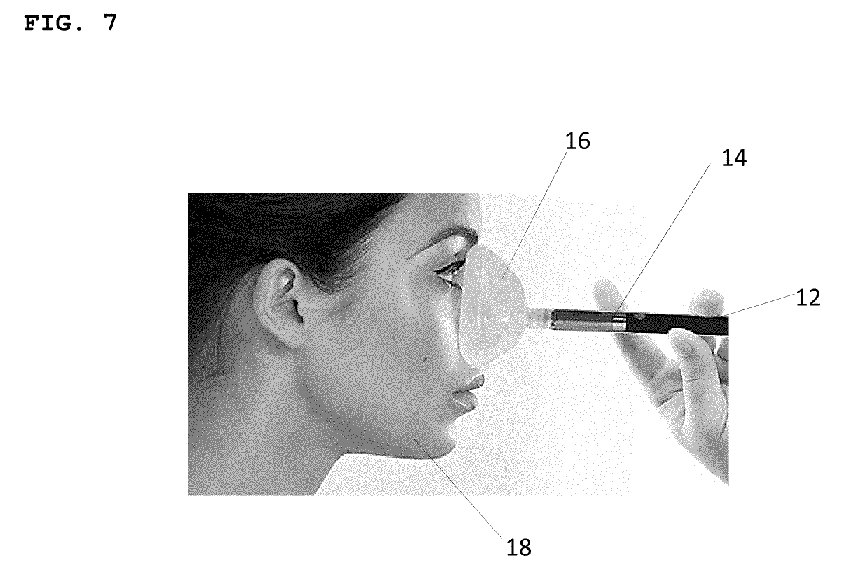

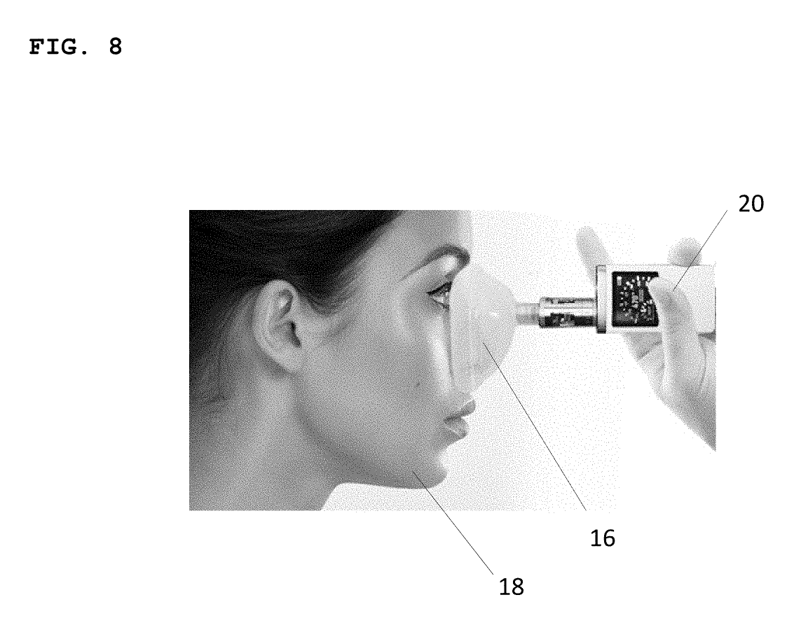

FIG. 7 illustrates a side view of one embodiment of the nasal adapter with the face mask mechanism in use. Herein, a user 18 is utilizing the nasal adapter with a battery device 12, an atomizer 14, and a face mask 16. In FIG. 8 the user 18, utilizes another embodiment of the nasal adapter wherein a digital read out atomizer 20, which allows the user to adjust temperature, vaporization, and other qualities for a customized use depending on the substance used. When in use, a user places the face mask over his nose area, activates the atomizer, and breathes in the released vapor.

In a preferred embodiment, the face mask mechanism 16 provides a one-way valve, which allows for a dispensing of mist and vapor that would be inhaled by the user 18.

In another embodiment, the battery device is removable so that when exhausted, a fresh dry battery may be substituted.

A list of some herbals and nutraceuticals without regulatory requirements that may be administered through the instant system include, but are not limited to the following:

1) Lung Cleansing Kit (Menthol including the active ingredients of VicsVaporub)
2) Weight Control (Active ingredients of Hydroxicut) Sleep Aid (Melatonin, diphenhydramine and other OTC drugs)
3) Anxiety, depression, relaxant (Valerian Root Extract with or without Bay Leaf Extract)
4) Energy (Formulations used in HealthCare, sold in Whole food stores)
5) CoQ10 (High priced formulations sold in GNC etc.)
6) Pain (Naproxen, Ibuprofen other NSAIDs)
7) Sleep Aid (melatonin, diphenhydramine and other OTCs)
8) Migraine (will look up a Nutraceutical or Herbal)
For Prescription Drugs specifically
1) Migraine (sumatriptan)

### 2) Viagra

The system may further comprise variations such as the use of Lavender for the purposes of:
a. preventing panic attack and anxiety;
b. calming effect aids relaxation;
c. reducing stress; and,
d. relieving pain from tension headache.

It should be understood that numerous variations are possible, while adhering to the inventive concept. Such variations are contemplated as being a part of the present invention.

What is claimed is:

1. A nasal adapter comprising:
a battery device;
a atomizer; and
a face mask mechanism.

2. A nasal adapter of claim 1 wherein the face mask mechanism fits over a user's nose.

3. A nasal adapter of claim 1, wherein the atomizer comprises a vaporized lunch cleansing system.
